(19) United States
(12) Patent Application Publication
(10) Pub. No.: US 2014/0200600 A1
(43) Pub. Date: Jul. 17, 2014

(54) EAR BRUSH CLEANER

(71) Applicant: Marc Blatstein, Potomac, MD (US)

(72) Inventor: Marc Blatstein, Potomac, MD (US)

(21) Appl. No.: 14/100,459

(22) Filed: Dec. 9, 2013

Related U.S. Application Data
(60) Provisional application No. 61/752,081, filed on Jan. 14, 2013.

(51) Int. Cl. A61F 11/00

(52) U.S. Cl. CPC A61F 11/006
USPC 606/162

(57) ABSTRACT

Ear Brush Cleaner© drawings based on an over the counter [OTC] interdental floss instrument. The ‘Flair or Flange’ concept: Ear Brush Cleaner© is to have a flair or flange in function similar to the one I've shown in my drawings that restricts its insertion into the ear canal, to be at a distance as to not contact the ear drum. The bristle filaments are to be flexible and soft as to not cause soft tissue damage as well as offering the option of combining an absorbent capability should that option be desired. This, along with ‘our sensation of proprioception’ adds yet another layer of protection to the inner ear. All of the attributes of the Ear Brush Cleaner© with its Flair or flange, makes this the only practical, safe and effective product for the removal of ear wax from the ear canal without contributing to injury of the ear drum.

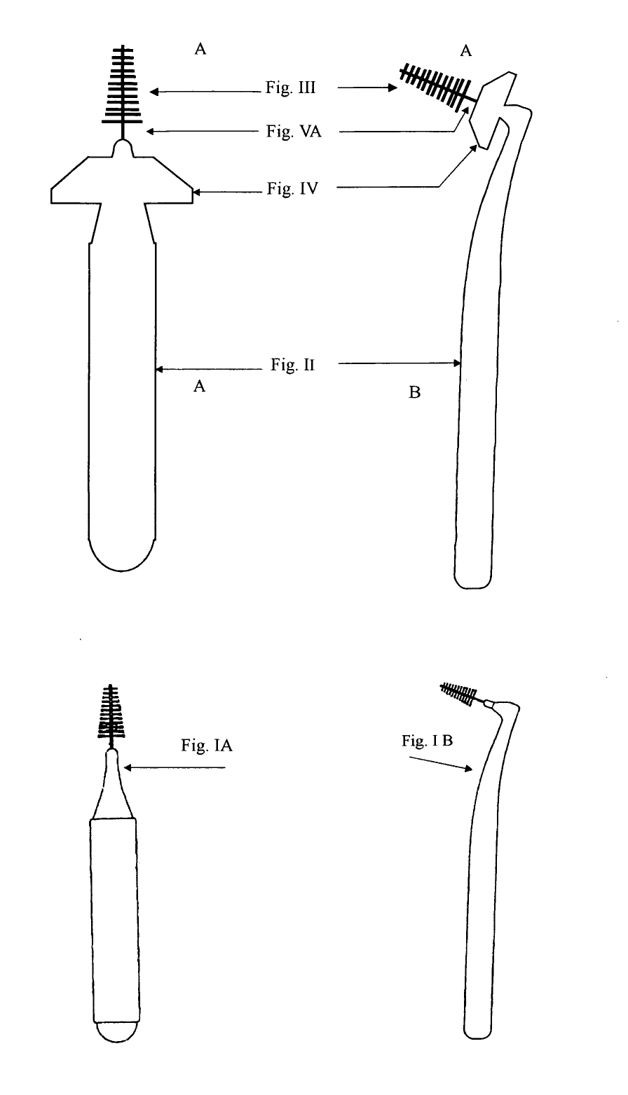

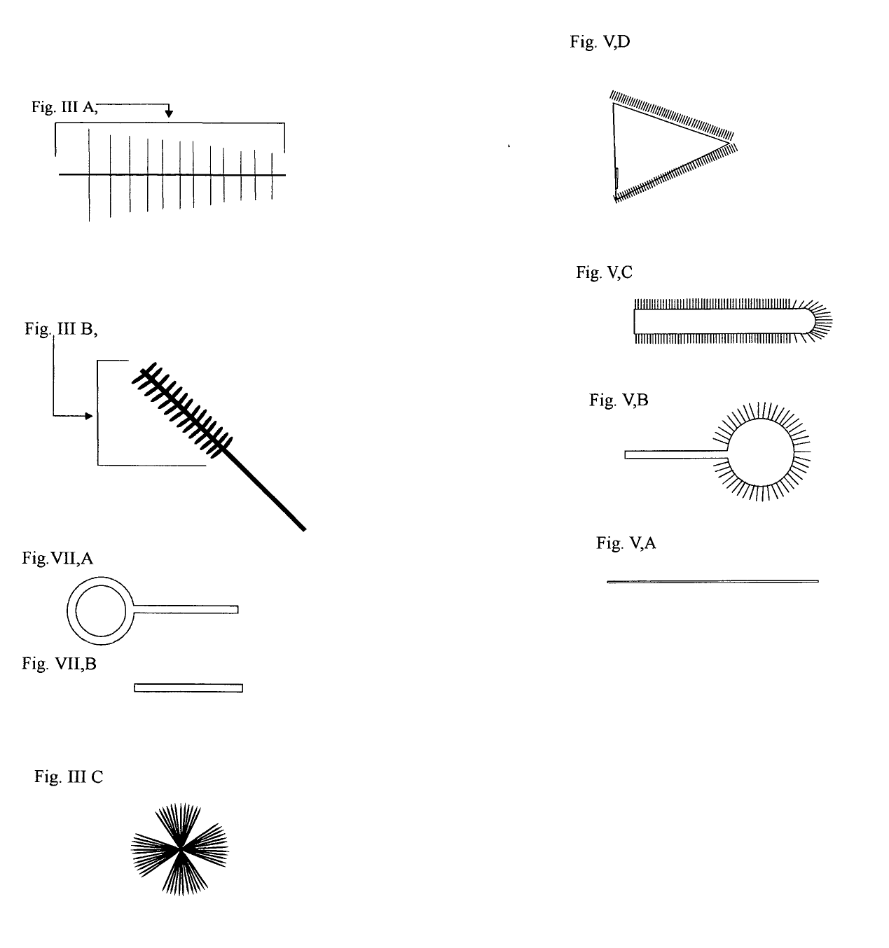

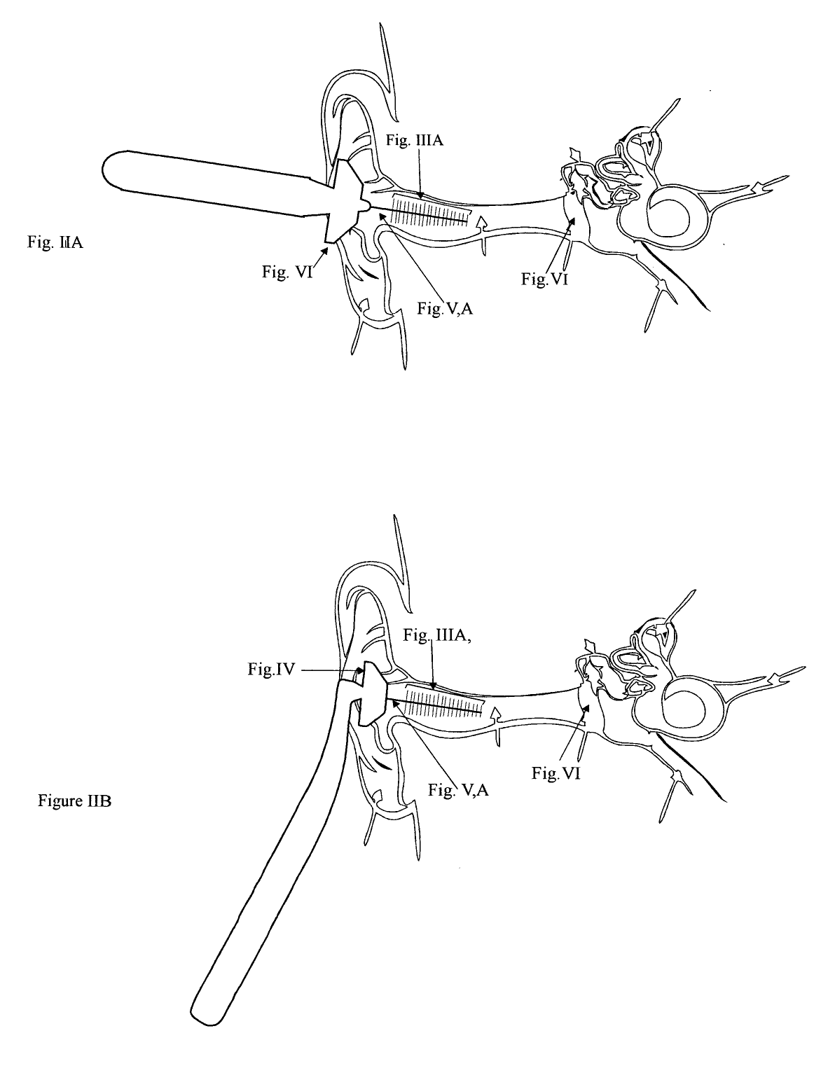

This invention takes the idea of an floss brush instrument, and adjusts it to be the only OTC product available for the practicable removal of ear wax safely. The present invention is compact, easy to carry (practical to use) as well as allowing it to be manufactured at low manufacturing cost.

### BACKGROUND OF THE INVENTION

Like most people, when I have had either an ear “itch”, or I felt that I needed to clean my ear of ear wax, I chose one of the following instruments: a paperclip, the end of a pen, a Q-tip, or my eye glasses (the end that hooks around the ear).

Then several years ago, while trying to get the ear wax out of my ear using one of the previously mentioned examples, I severely impacted the wax further into my ear thus creating a very Painful Problem. I made an appointment with an ENT colleague who explained to me the dynamics of what happened and proceeded to saline flush out my ear. I vowed never to go through this again.

From colleagues and friends alike, those who wear hearing aids and those who don't, I've learned that wax Buildup and its removal usually involves an unexpected visit to the doctor. I asked colleagues the following question: What do you use to clean out your ears? Q-tips were the most common answer (even with their disclaimers not to do so). Only trained ENTs knew that these should not be used as they could contribute to injury to the ear drum and/or impacting ear wax within the canal.

Over the following weeks I was on a mission, and that was over 3 years ago.

While using an OTC (Over the Counter) interdental instrument to floss, I experimented and took out a fresh one to clean out my ears. While the bristles were too stiff, it had worked! Out came the ear wax while at the same time I scratched my itchy area. Since that moment I have used similar type interdental instrument to clean my ears. In the following years I looked at every ear care product that was available OTC. They consisted of either cumbersome liquids or oils that one squirts into one's ear, or rigid devices (that could also damage one's eardrum), while at the same time were not useful, safe or practical. One very popular product even had a disclaimer—“To Not” put them into your ear; however most of us still continued to do so.

Recently an ear cleaner that has a protective flange and uses a vacuum to suck out ear wax, foreign debris as well as liquids has come to market. While it appears to function very efficiently and safely, its inherent limitation is that it is to large & bulky to be carried throughout the day during our ‘activities of daily living’.

The ear brush will occupy less space and can be easily carried in a pocket or a handbag. The Figures demonstrate several different handle combinations with varying brush type bristle filament combinations. A cap, not pictured can be added to cover the brush bristle filament portion to help it from being further contaminated. The users hold the handle and insert the brush portion into the ear, allowing the flair/flange and or flange with shaped handle to protect the eardrum by preventing its insertion into the ear canal.

During our ‘activities of daily living’, even though most of us are often away from home, these brushes can be easily taken with us used & then cleaned after each use. Should the cap be included, they can then be re-capped, thus allowing them to be re-used multiple times.

In Summary, the Ear Brush Cleaner© has ‘my flair’ that prevents eardrum contact thus providing a device that can be used to safely clean ones ear. The bristle filaments are to be flexible and soft as to not cause soft tissue damage as well as offering the option for some of the filaments to be absorbent, flexible and yet ridged enough to facilitate ear wax penetration and removal.

### OBJECTS AND SUMMARY OF THE INVENTION

One object of this invention is to provide a safe & practical way to remove ear wax from the ear.

Another object of this invention is to have flair or flange so as to prohibit the brush bristle filament component from abutting the eardrum.

Another object of this invention is to have bristles of such tensile flexibility as to not cause soft tissue injury or damage to the ear drum.

Another object of this invention is to have the most optimal bristle shaped filament to facilitate ear wax removal.

Another object of this invention is to have the most optimal bristle shape filament & material to facilitate liquidly fluid removal should this benefit be required.

Another object of this invention is to have its handle to act alone or as part of the flair-flanges purpose in prohibiting the brush-bristle abutment against the eardrum.

### BRIEF DESCRIPTIONS OF THE DRAWINGS

Referring to the figures of the brush assembly, the present invention comprises a plurality of brushes with each having a Handle, Flair (Flange), Neck and Filament.

Figure IA; Prior Art, Handle Straight Interdental Brush

Figure IB; Prior Art, Handle Angled Interdental Brush

Figure IIA; New Flair or Flange with Handle Straight

Figure IIB; New Flair or Flange with Handle Angled

Figures IIA and B (Handle options), using the handle, the Ear Brush Cleaner is manually rotated as debris and wax is scoured out of the ear.

Figure IIIA; Triangle overall shaped filaments

Figure IIIB; Straight or Rectangular overall shaped filaments

Figure IIIC; Spindle overall shaped filaments

Figure IIIA-IIIC; Filament ‘overall shape’ options, individually or in combination, to be of an appropriate size, length, stiffness and yet flexibility, appropriate to the needs of the invention.

Figure IV; Flair or Flange, prohibits the Ear Drum and Ear Brush cleaner from coming into contact with each other.

Figure V,A; the Neck Thin

Figure V,B; the Neck Ball Shaped

Figure VC; the Neck Rectangular with convexed end

Figure VC; the Neck Triangle

Figure VA-VD; The Neck (Options) is where the filaments are attached, all to be of an appropriate size, length, and material appropriate to the needs of the invention

Figure VI; Ear Drum

Figure VII, A: Individual filament made with a Loop, follows a medical loop instrument similar to what is currently used by the medical community.

Figure VII,B: Individual filament, straight

Figures VII, A and B; New Filaments Options: to be of an appropriate size, length, and material appropriate to the needs of the invention. Made in the non-absorbable, as well as having the ability to absorb liquid should that be of benefit to the invention.

The foregoing descriptions should be considered as illustrative only of the principles of the invention. Since numerous modifications and changes will readily occur to those skilled in the art, it is not desired to limit the invention to the exact construction and operation shown and described, and, accordingly, all suitable modifications and equivalents may be resorted to, falling within the scope of the invention.

### EMBODIMENTS OF THE INVENTION

Referring to the figures of the brush assembly, the present invention comprises a plurality of brushes with each having a handle, a brush portion that is connected to an extension, extending from an end of the handle. Each handle includes two flat surfaces as to form a potential holding area, and ridges that extend from an outer surface of the holding area. The ridges are oriented to be perpendicular to a longitudinal axis of the handle such that the user can easily hold the ear brushes.

The handles may be able to be made either lying individually or having connections with each other side by side by connection plates and each connection plate having a notch defined in a top edge thereof so that the user can conveniently tear the connection plate apart to pick one of the ear brushes. Should those skilled in the art demonstrate that a different method (or no method) of attachment be best, here too all suitable modifications and equivalents may be resorted to, falling within the scope of the invention.

Safety to the ear drum is further accomplished by providing the Ear Brush Cleaner in appropriate varying lengths, where the package labeling will notify the user as to whether they are for adult or child usage along with other qualifying descriptive language and instructions standard in the industry today. A container is provided to receive the brushes, with packaging designed to be easily opened so that the user can pick one of the brushes easily. The ear brushes may be connected side by side by the connection plates, or laid out side by side so that they are well organized in the container. Both the brush &/or container occupy a small enough space allowing the user to carry them in pockets, thus making them easily available to be used during the activities of daily living.

Each handle of the brushes has a holding area for convenience of grasp and the connection plates allow the user to pick the brushes one by one, easily.

Parts of the brushes can be produced in mass production by way of plastic injection allowing for a low manufacturing cost.

While we have shown and described the embodiment in accordance with the present invention, it should be clear to those skilled in the art that further embodiments may be made without departing from the scope of the present invention.

In describing a preferred embodiment of the invention illustrated here, specific terminology will be resorted to for the sake of clarity. However, the invention is not intended to be limited to the specific terms so selected & described here, it is to be understood that each specific term includes all technical equivalents which operate in a similar manner to accomplish a similar purpose.

The foregoing description should be considered as illustrative only of the principles of the invention. Since numerous modifications and changes will readily occur to those skilled in the art, it is not desired to limit the invention to the exact construction and operation shown and described, and, accordingly, all, suitable modifications and equivalents may be resorted to, falling within the scope of the invention.

1. I claim a device for cleaning out debris in the external ear canal.

2. I claim, according to claim 1, when inserted into the external ear canal is rotated by hand manipulation to scour out the debris, a device having a variety of tip structure options that will scour out the ear canal, without irritating or injuring the soft tissue walls of the ear canal or abutting the eardrum.

3. I claim a device for cleaning out debris in the external ear canal that protects the eardrum

4. I claim, according to claim 3, the flange or flair with handle that will prohibit the device from abutting and injuring the eardrum, a device that has a circumferential flange that acts a safety mechanism to prevent too great an insertion into the ear and that that provides a practical way to clean ones ear safely during the normal activities of daily living.

5. I claim a device that with the addition of a cap may provide the option for it to be reused.
